(19) United States
(12) Patent Application Publication
(10) Pub. No.: US 2006/0229242 A1
(43) Pub. Date: Oct. 12, 2006

(54) Composition comprising a pulmonary surfactant and a pde2 inhibitor

(75) Inventor: Stefan-Lutz Wollin, Meersburg (DE)

Correspondence Address: NATH & ASSOCIATES PLLC, 112 South West Street, Alexandria, VA, 22314, US

(73) Assignee: Altana Phazma AG, Konstanz (DE)

(21) Appl. No.: 10/568,817

(22) PCT Filed: Aug. 27, 2004
(86) PCT No.: PCT/EP04/51948

§ 371 Date: Feb. 21, 2006

(30) Foreign Application Priority Data
Aug. 28, 2003 (EP) 03019447.6

(51) Int. Cl. A61K 38/17; A61K 31/522; A61K 31/52; A61K 31/519

(52) U.S. Cl. 514/12; 514/263.3; 514/263.22; 514/263.4; 514/262.1

(57) ABSTRACT

The invention relates to the combined administration of a pulmonary surfactant and a PDE2 inhibitor for the treatment of a disease in which pulmonary surfactant malfunction and/or phosphodiesterase 2 (PDE2) activity is detrimental.

### FIELD OF APPLICATION OF THE INVENTION

The invention relates to the combination of certain known active compounds for therapeutic purposes. The compounds used in the combination according to this invention are known pulmonary surfactants and known active compounds from the phosphodiesterase 2 (PDE2) inhibitor class. Their combined use in the sense according to this invention for therapeutic purposes has not yet been described in prior art.

### PRIOR ART

ARDS (Adult Respiratory Distress Syndrome) is a descriptive expression which is applied to a large number of acute, diffuse infiltrative pulmonary lesions of differing etiology if they are associated with a severe gas exchange disorder (in particular arterial hypoxemia) [G. R. Bernard et al.: Report of the American-European consensus conference on ARDS: definitions, mechanisms, relevant outcomes and clinical trial coordination; Intensive Care Medicine, 1994, 20:225232]. The expression ARDS is also used for IRDS (Infant Respiratory Distress Syndrome) because of the numerous common clinical and pathological features. If, in the case of IRDS, the lung surfactant deficiency caused by premature birth is predominant, then in the case of ARDS a lung surfactant malfunction is caused by the disease of the lung based on differing etiologies such as inhalation of toxins or irritants (e.g. chlorine gas, nitrogen oxides, smoke), direct or indirect trauma (e.g. multiple fractures or pulmonary contusion), systemic reactions to inflammations outside the lung (e.g. hemorrhagic pancreatitis, gram-negative septicemia), transfusions of high blood volumes or alternatively after cardiopulmonary bypass. In patients suffering from ARDS, lung surfactant function is impaired (=surfactant malfunction) so that the alveolar surfactant layer does not prevent lung atelectasis and does not maintain physiologic lung functions required for oxygenation.

In the healthy lung, pulmonary endothelium regulates the exchange of fluid, solutes, macromolecules, and cells between vascular and tissue spaces. With inflammation abound in ARDS, the endothelial barrier becomes more permissive for exchange leading to interstitial and alveolar edema formation. This process leads to a further impairment of oxygenation.

Presently, the therapy of ARDS mainly consists in the earliest possible application of different forms of ventilation (e.g. raising of the oxygen concentration of the respiratory air) up to extracorporeal membrane oxygenation. The specific use of various ventilation techniques has only led to a small lowering of mortality and including the risk of damaging the lungs by ventilation with pressure and high FiO₂ (Fraction of Inspired Oxygen; proportion of oxygen in the respiratory air). In particular, ARDS patients whose lungs have been damaged by ventilation need even higher pressures and higher FiO₂ to obtain an adequate oxygenation of the blood.

Because surfactant function is impaired in ARDS, surfactant replacement therapy is thought to improve lung function and oxygenation in ARDS. It has also proven suitable to treat IRDS by introducing pulmonary surfactant preparations into the lungs of the children concerned. WO01076619 describes the use of a pulmonary surfactant preparation for the prophylaxis or early treatment of acute pulmonary diseases such as ARDS, IRDS or ALI (Acute Lung Injury). WO03033014, Spragg R G et al. [Spragg R G et al. (2003) American Journal Respiratory and Critical Care Medicine 167: 1562] and Eaton S et al. [Eaton S et al. (2002) Expert Opinion on Investigational Drugs 11: 37] describe that pulmonary surfactants, in particular rSP-C surfactants, are useful in the treatment of ARDS.

Asthma patients, in particular in acute status asthmaticus, suffer from obstructed airways due to bronchoconstriction, inflammation, mucus hypersecretion, and edema formation. Due to extravasation of plasma and proteins into the alveolar lumen and due to released proteases, and mucus the surfactant function is disturbed leading to atelectasis and impaired ventilation [Hohlfeld J M et al. Dysfunction of pulmonary surfactant in asthmatics after segmental allergen challenge. Am J Respir Crit Care Med 1999; Fuchimukai T et al. Artificial pulmonary surfactant inhibited by proteins. J Appl Physiol 1987, 62:429-437: Seeger W et al. Surfactant inhibition by plasma proteins: differential sensitivity of various surfactant preparations. Eur Respir J 1993, 6:971-977]. Fatal asthma attacks end up with insufficient oxygenation resulting partly from edema formation and impaired ventilation due to a lack of active surfactant.

There is first evidence on the value of surfactant treatment of patients with Asthma in a pilot study the patients were treated with surfactant inhalation after an asthma attack. Respiratory functions and oxygenation were markedly improved in all patients [Kurashima Ket al. A pilot study of surfactant inhalation in the treatment of asthmatic attack. Arerugi. 1991 February ;40(2):160-3].

WO 01058423 describes the use of pulmonary surfactant for the prophylaxis or treatment of chronic pulmonary diseases in mammals such as chronic obstructive pulmonary disease (COPD), asthma, cystic fibrosis, pulmonary fibrosis, pulmonary degeneration, chronic bronchitis and pulmonary emphysema.

Recent data show that PDE2 is one of the major enzymes found in bovine and porcine endothelial cells [Ashikaga T et al. Altered expression of cyclic nucleotide phosphodiesterase isozymes during culture of aortic endothelial cells Biochem Pharmacol. 1997 Nov. 15;54(10):1071-9; Kishi Yet al. Phosphodiesterases in vascular endothelial cells. Adv Second Messenger Phosphoprotein Res. 1992; 25:201-13; Koga S et al. TNF modulates endothelial properties by decreasing CAMP. Am J Physiol. 1995 May; 268(5 Pt 1):C1104-13; Lugnier C, Schini V B. Characterization of cyclic nucleotide phosphodiesterases from cultured bovine aortic endothelial cells. Biochem Pharmacol. 1990 Jan 1; 39(1):75-84; Souness J E et al. Pig aortic endothelial-cell cyclic nucleotide phosphodiesterases. Use of phosphodiesterase inhibitors to evaluate their roles in regulating cyclic nucleotide levels in intact cells. Biochem J. 1990 Feb 15;266(1):127-32]. Inhibition of PDE2 reduces monolayer permeability of porcine pulmonary artery endothelial cells [Suftorp N et al. Role of nitric oxide and phosphodiesterase isoenzyme II for reduction of endothelial hyperpermeability. Am J Physiol. 1996 March;270; (3 Pt 1):C778-85]. Finally, Suttorp N. et al. [Suttorp N. et al. (1996) Atemwegs und Lungenkrankheiten, DustriVerlag, Vol. 22: 560-566] describes the use of PDE2 inhibitors to block pulmonary vascular leakage.

In the European patent application EP 0771799, the international patent application WO98/40384 and in the U.S. Pat. No. 5,861,396 purin-6-one derivatives are described as PDE2 inhibitors suitable for the treatment of cardiovascular disorders, disorders of the vascular system and of the urogenital system.

### SUMMARY OF THE INVENTION

It is the object of the present invention to make available a pharmaceutical composition suited for prevention or reduction of the onset of symptoms of a disease, or for treatment or reduction of the severity of a disease in which pulmonary surfactant malfunction and/or phosphodiesterase 2 (PDE2) activity is detrimental.

Surprisingly, it has now been found that the combined use of a pulmonary surfactant and a PDE2 inhibitor fulfills these conditions.

Thus, the invention relates to pharmaceutical compositions comprising a pulmonary surfactant in combination with a PDE2 inhibitor and to methods for preventing or reducing the onset of symptoms of a disease in which pulmonary surfactant malfunction and/or phosphodiesterase 2 (PDE2) activity is detrimental, and to methods for treating or reducing the severity of a disease in which pulmonary surfactant malfunction and/or phosphodiesterase 2 (PDE2) activity is detrimental.

Accordingly, the invention relates in a first aspect to the combined use of a pulmonary surfactant and a PDE2 inhibitor for preventing or reducing the onset of symptoms of a disease, or treating or reducing the severity of a disease in a patient in need thereof, in which disease pulmonary surfactant malfunction and/or phosphodiesterase 2 (PDE2) activity is detrimental.

In another aspect of present invention, there is provided the use of a combination of a pulmonary surfactant and a PDE2 inhibitor for the preparation of a medicament for preventing or reducing the onset of symptoms of a disease, or treating or reducing the severity of a disease in a patient in need thereof, in which disease pulmonary surfactant malfunction and/or phosphodiesterase 2 (PDE2) activity is detrimental.

In another aspect of present invention, there is provided a method for preventing or reducing the onset of symptoms of a disease in which pulmonary surfactant malfunction and/or phosphodiesterase 2 (PDE2) activity is detrimental, or treating or reducing the severity of a disease in which pulmonary surfactant malfunction and/or phosphodiesterase 2 (PDE2) activity is detrimental by administering to a patient in need thereof an effective amount of (1) a pulmonary surfactant and (2) a PDE2 inhibitor.

In another aspect of present invention, there is provided a method for preventing or reducing the onset of symptoms of a disease in which pulmonary surfactant malfunction and/or phosphodiesterase 2 (PDE2) activity is detrimental, or treating or reducing the severity of a disease in which pulmonary surfactant malfunction and/or phosphodiesterase 2 (PDE2) activity is detrimental by simultaneously administering to a patient in need thereof an effective amount of a pulmonary surfactant and a PDE2 inhibitor.

In another aspect of present invention, there is provided a method for preventing or reducing the onset of symptoms of a disease in which pulmonary surfactant malfunction and/or phosphodiesterase 2 (PDE2) activity is detrimental, or treating or reducing the severity of a disease in which pulmonary surfactant malfunction and/or phosphodiesterase 2 (PDE2) activity is detrimental by administering in succession, close in time or remote in time, in any order whatever to a patient in need thereof an effective amount of a pulmonary surfactant and a PDE2 inhibitor.

In another aspect of present invention, there is provided a pharmaceutical composition suited for a method for preventing or reducing the onset of symptoms of a disease in which pulmonary surfactant malfunction and/or phosphodiesterase 2 (PDE2) activity is detrimental, or for treating or reducing the severity of a disease in which pulmonary surfactant malfunction and/or phosphodiesterase 2 (PDE2) activity is detrimental, which pharmaceutical composition comprises a combination of an effective amount of a pulmonary surfactant and an effective amount of a PDE2 inhibitor.

In another aspect of present invention, there is provided a pharmaceutical composition suited for a method for preventing or reducing the onset of symptoms of a disease in which pulmonary surfactant malfunction and/or phosphodiesterase 2 (PDE2) activity is detrimental, or for treating or reducing the severity of a disease in which pulmonary surfactant malfunction and/or phosphodiesterase 2 (PDE2) activity is detrimental, which pharmaceutical composition comprises as a fixed combination an effective amount of a pulmonary surfactant and an effective amount of a PDE2 inhibitor, and optionally a pharmaceutically acceptable carrier. In particular, such a fixed pharmaceutical composition for intratracheal or intrabronchial instillation is preferred.

In another aspect of present invention, there is provided a pharmaceutical composition suited for a method for preventing or reducing the onset of symptoms of a disease in which pulmonary surfactant malfunction and/or phosphodiesterase 2 (PDE2) activity is detrimental, or for treating or reducing the severity of a disease in which pulmonary surfactant malfunction and/or phosphodiesterase 2 (PDE2) activity is detrimental, which pharmaceutical composition comprises as a free combination an effective amount of a pulmonary surfactant and optionally a pharmaceutically acceptable carrier and an effective amount of a PDE2 inhibitor and optionally a pharmaceutically acceptable carrier.

In another aspect of present invention there is provided the use of a pharmaceutical composition comprising a combination of a pulmonary surfactant and a PDE2 inhibitor for the treatment of ALI, ARDS, IRDS or Asthma bronchiale.

In another aspect of present invention there is provided the use of a combination of a pulmonary surfactant and a PDE2 inhibitor for the preparation of a medicament for the treatment of ALI, ARDS, IRDS or Asthma bronchiale.

In another aspect of present invention there is provided a method for preparing a pharmaceutical composition suited for preventing or reducing the onset of symptoms of a disease in which pulmonary surfactant malfunction and/or phosphodiesterase 2 (PDE2) activity is detrimental, or for treating or reducing the severity of a disease in which pulmonary surfactant malfunction and/or phosphodiesterase 2 (PDE2) activity is detrimental, which method comprises the step: mixing an effective amount of a pulmonary surfactant and a PDE2 inhibitor with a pharmaceutically acceptable carrier.

### DETAILED DESCRIPTION OF THE INVENTION

The combination therapy, which is the subject matter of present invention, comprises administering a pulmonary surfactant with a PDE2 inhibitor to prevent the symptoms or the onset of a disease or to treat a disease in which pulmonary surfactant malfunction and/or phosphodiesterase 2 (PDE2) activity is detrimental.

The invention thus relates to the combined use of a pulmonary surfactant and a PDE2 inhibitor in preventing the symptoms of, or treating a disease in which pulmonary surfactant malfunction and/or phosphodiesterase 2 (PDE2) activity is detrimental.

The “pulmonary surfactant” useful in this invention may be any compound or pulmonary surfactant preparation that is known to have the same surface-active properties as natural pulmonary surfactant; natural pulmonary surfactant reduces, for example, the surface tension in the alveoli.

A simple and rapid in vitro test with which the surface activity of pulmonary surfactant can be determined is, for example, the so-called Wilhelmy balance [Goerke, J. Biochim. Biophys. Acta, 344: 241-261 (1974), King R. J. and Clements J. A., Am. J. Physicol. 223: 715-726 (1972)]. This method gives information on the pulmonary surfactant quality, measured as the action of a pulmonary surfactant of achieving a surface tension of almost zero mN/m. Another measuring device for determining the surface activity of pulmonary surfactant is the pulsating bubble surfactometer [Possmayer F. et al., Prog. Resp. Res., Ed. v. Wichert, Vol. 18: 112-120 (1984)]. The activity of a pulmonary surfactant preparation can also be determined by means of in vivo tests, for example as described by Häfner et al. [D. Häfner et al.: Effects of rSP-C surfactant on oxygenation and histology in a rat lung lavage model of acute lung injury. Am. J. Respir. Crit. Care Med. 1998,158: 270-278].

A group of known pulmonary surfactant preparations and their modifications that may be usefully as pulmonary surfactant employed in the present invention include pulmonary surfactant preparations having the function of natural pulmonary surfactant. Preferred pulmonary surfactant preparations are those which, for example, have activity in the tests described above. Particularly preferred pulmonary surfactant preparations are those which exhibit increased activity in such a test in comparison with natural, in particular human, pulmonary surfactant. In this context, these can be compositions which only contain phospholipids, but also compositions which, apart from the phospholipids, inter alia additionally contain pulmonary surfactant protein.

Preferred phospholipids according to the invention are dipalmitoylphosphatidylcholine (DPPC), palmitoyloleylphosphatidylglycerol (POPG) and/or phosphatidylglycerol (PG). Particularly preferably, the phospholipids are mixtures of various phospholipids, in particular mixtures of dipalmitoylphosphatidylcholine (DPPC) and palmitoyloleylphosphatidylglycerol (POPG), preferably in the ratio from 7 to 3 to 3 to 7.

Commercial products which may be mentioned as pulmonary surfactant preparations are
CUROSURF® (INN: PORACTANT ALFA) (Serono, Pharma GmbH, Unterschleiβheim), a natural surfactant from homogenized porcine lungs;
SURVANTA® (INN: BERACTANT) (Abbott GmbH, Wiesbaden), extract of bovine lungs;
ALVEOFACT® (INN: BOVACTANT) (Boehringer Ingelheim), extract of bovine lungs;
EXOSURF® (INN: COLFOSCERIL PALMITATE) (Glaxo SmithKline), a synthetic phospholipid containing excipients;
SURFACTEN® (INN: SURFACTANT-TA) (Mitsubishi Pharma Corporation), a pulmonary surfactant extracted from bovine lungs;
INFASURF® (INN: CALFACTANT) (Forest Pharmaceuticals), a surfactant extracted from calf lungs;
ALEC® (INN: PUMACTANT) (Britannia Pharmaceuticals), an artificial surfactant of DPPC and PG; and
BLES® (BLES Biochemical Inc.), a bovine lipid extract surfactant.

Suitable pulmonary surfactant proteins are both the proteins obtained from natural sources, such as pulmonary lavage or extraction from amniotic fluid, and the proteins prepared by genetic engineering or chemical synthesis. According to the invention, in particular the pulmonary surfactant proteins designated by SP-B (Surfactant Protein-B) and SP-C (Surfactant Protein-C) and their modified derivatives are of interest. The amino acid sequences of these pulmonary surfactant proteins, their isolation or preparation by genetic engineering are known (e.g. from WO 8603408, EP 0251449, WO 8904326, WO 8706943, WO 8803170, WO 9100871, EP 0368823 and EP 0348967). Modified derivatives of the pulmonary surfactant proteins designated by SP-C, which differ from human SP-C by the replacement of a few amino acids, are described, for example, in WO 9118015 and WO 9532992. Particularly to be emphasized in this connection are the recombinant SP-C (rSP-C) derivatives which are disclosed in WO 9532992, in particular those which differ from human SP-C in positions 4 and 5 by the substitution of cysteine by phenylalanine and in position 32 by the substitution of methionine by isoleucine [designated herein as rSP-C (FF/I) or LUSUPULTIDE (INN) or VENTICUTE®]. Modified derivatives of pulmonary surfactant proteins are also understood as meaning those proteins which have a completely originally designed amino acid sequence with respect to their pulmonary surfactant properties, such as are described in EP 0593094 and WO 9222315. Preferably, the polypeptide KL4 (INN: SINAPULTIDE, SURFAXIN®) may be mentioned in this connection. The name pulmonary surfactant protein, according to the invention, also comprises mixtures of different pulmonary surfactant proteins. In EP 0100910, EP 0110498, EP 0119056, EP 0145005 and EP 0286011 phospholipid compositions with and without pulmonary surfactant proteins are described which are likewise suitable as components of the preparations.

As further constituents which can be present in pulmonary surfactant preparations, fatty acids such as palmitic acid may be mentioned. The pulmonary surfactant preparations can also contain electrolytes such as calcium, magnesium and/or sodium salts (for example calcium chloride, sodium chloride and/or sodium hydrogencarbonate) in order to establish an advantageous viscosity. Preferred pulmonary surfactant preparations according to the invention contain 80 to 95% by weight of phospholipids, 0.5 to 3.0% by weight of pulmonary surfactant proteins, 3 to 15% by weight of fatty acid, preferably palmitic acid, and 0 to 3% by weight of calcium chloride.

The pulmonary surfactant preparations are prepared by processes known per se and familiar to the person skilled in the art, for example as described in WO 9532992. According to the invention, the pulmonary surfactant preparations are preferably lyophilized and in particular spray-dried pulmonary surfactant preparations. Lyophilized preparations are disclosed, for example, in WO 9735882, WO 9100871 and DE 3229179. WO 9726863 describes a process for the preparation of powdered pulmonary surfactant preparations by spray drying. According to the invention, preparations prepared in this way are preferred.

According to this invention, the term “PDE2 inhibitor” refers to a selective phosphodiesterase (PDE) inhibitor, which inhibits preferentially the type 2 phosphodiesterase (PDE2) when compared to other known types of phosphodiesterase, e.g. type 1, 3, 4, 5, etc. (PDE1, PDE3, PDE4, PDE5, etc.). According to this invention, a PDE inhibitor preferentially inhibiting PDE2 refers to a compound having a lower IC50 for PDE2 (i.e. the IC50 for PDE2 inhibition is about 10 times lower than the IC50 for inhibition of other known types of phosphodiesterase, e.g. type 1, 3, 4, 5, etc.) and therefore is more potent to inhibit PDE2.

For activity determination of PDE2, the [³H]CAMP SPA assay (Amersham Life Science) may be used −10⁻⁶M cGMP being added to the reaction mixture to activate the enzyme. Other methods for activity testing of PDE2 inhibitors are disclosed in WO 9840384 and U.S. Pat. No. 5,861,396. It is also possible to determine PDE2 activity by the method described by Tenor H et al. [Tenor H et al. (2002) British J Pharmacol 135: 609].

A group of PDE2 inhibitors that may be usefully employed in present invention include the purin-6-one derivatives as revealed in EP 0771799, WO98/40384 and in U.S. Pat. No. 5,861,396.

Compounds which may be mentioned as preferred examples of PDE2 inhibitors are
N-Benzyl-2-[5-fluoro-2-methyl-1(Z)-(3,4,5-trimethoxybenzylidene)inden-3-yl]acetamide,
2-(3′-Aminobiphenyl-4-ylmethyl)-9-(1-methyl-4-phenylbutyl)hypoxanthine,
N-Benzyl-2-[5-fluoro-2-methyl-1(Z)-(3,4,5-trimethoxybenzylidene)inden-3-yl]acetamide,
2-(3′-Aminobipheny-4-ylmethyl)-9-(1-methyl-4-phenylbutyl)hypoxanthine,
2-Benzyl-9-(1-methyl-4-phenylbutyl)hypoxanthine,
2-(3,4-Dichlorobenzyl)-9-[1-(1-hydroxyethyl)-4-phenylbutyl]hypoxanthine,
2-(4-Fluorobenzyl)-9-(1-methyl-4-phenylbutyl)hypoxanthine,
9-(1-Methyl-4-phenylbutyl)-2-[4-(3-thienyl)benzyl]hypoxanthine,
1-[5-[9-[1-(1-Hydroxyethyl)-4-phenylbutyl]hypoxanthin-2-ylmethyl]-2-methoxyphenylsulfonyl]piperidine-4-carboxylic acid,
2-(Biphenyl-4-yl)-9-(1-methyl-4-phenylbutyl)-6,9-dihydro-1H-purin-6-one,
2-(4-Chlorophenyl)-9-[1-(1-hydroxyethyl)heptyl]-6,9-dihydro-1H-purin-6-one,
2-Cyclohexyl-9-[1-(1-hydroxyethyl)-4-phenylbutyl]-6,9-dihydro-1H-purin-6-one,
2-Cyclopropyl-9-(1-methyl-4-phenylbutyl)-6,9-dihydro-1H-purin-6-one,
2-(1,3-Benzodioxol-5-yl)-9-(1-methyl-4-phenylbutyl)-6,9-dihydro-1H-purin-6-one,
erythro-9-(2-hydroxy-3-nonyl)adenine,
9-(6-Phenyl-2-oxohex-3-yl)-2-(3,4-dimethoxybenzyl)-purin-6-one,
6-(3,4-Dimethoxy-benzyl)-1-[1-(1-hydroxy-ethyl)-4-phenyl-butyl]-3-methyl-1,5-dihydro-pyrazolo[3,4-d]pyrimidin-4-one,
N-benzyl-2-(6-fluoro-2-methyl-3-pyridin-4-ylmethylene-3H-inden-1-yl)-acetamide,
(1Z)-N-benzyl-2-[6-fluoro-2-methyl-3-(3,4,5-trimethoxybenzylidene)-3H-inden-1-yl]-acetamide,
N-Benzyl-2-[5-fluoro-2-methyl-1-[(Z)-(pyridin-4-yl)methylene]-1H-inden-3-yl]acetamide hydrochloride,
4-[N-[4-[9-[N-Methyl-N-(3-phenylpropyl)amino]hypoxanthin-2-ylmethyl]phenyl]carbamoyl]piperidine-1-carboxylic acid benzyl ester,
4-[N-[4-[9-(N-hexyl-N-methylamino)hypoxanthin-2-ylmethyl]phenyl]carbamoyl]piperidine-1-carboxylic acid benzyl ester,
2-(3,4-Dimethoxybenzyl)-9-[N-methyl-N-(3-phenylpropyl)amino]hypoxanthine,
9-[N-Methyl-N-(3-phenylpropyl)amino]-2-[4-(4-methylpiperazin-1-ylsulfonyl)benzyl]hypoxanthine,
2-(3,4-Dimethoxybenzyl)-7-[1(R)-[1(R)-hydroxyethyl]-4-phenylbutyl]-5-methylimidazo[5,1-f][1,2,4]triazin-4(3H)-one,
7-(1-Acetylpentyl)-5-methyl-2-(4-methylbenzyl)imidazo[5,1-f][1,2,4]triazin-4(3H)-one,
7-(1-Acetylhexyl)-5-methyl-2-(4-methylbenzyl)imidazo[5,1-f][1,2,4]triazin-4(3H)-one,
2-(3,4-Dimethoxybenzyl)-7-[1-(1-hydroxyethyl)-5-hexenyl]-5-methylimidazo[5,1-f][1,2,4]triazin-4(3H)-one, and
and the pharmaceutically acceptable salts of these compounds.

Particularly preferred examples of PDE2 inhibitors are
erythro-9-(2-hydroxy-3-nonyl)adenine,
9-(6-Phenyl-2-oxohex-3-yl)-2-(3,4-dimethoxybenzyl)-purin-6-one,
6-(3,4-Dimethoxy-benzyl)-1-[1-hydroxy-ethyl)-4-phenyl-butyl]-3-methyl-1,5-dihydro-pyrazolo[3,4-d]pyrimidin-4-one,
N-benzyl-2-(6-fluoro-2-methyl-3-pyridin-4l-ylmethylene-3H-inden-1-yl)-acetamide,
(1Z)-N-benzyl-2-[6-fluoro-2-methyl-3-(3,4,5-trimethoxybenzylidene)-3H-inden-1-yl]-acetamide,
4-[N-[4-[9-[N-Methyl-N-(3-phenylpropyl)amino]hypoxanthin-2-ylmethyl]phenyl]carbamoyl]piperidine-1-carboxylic acid benzyl ester,
4-[N-[4-[9-(N-hexyl-N-methylamino)hypoxanthin-2-ylmethyl]phenyl]carbamoyl]piperidine-1-carboxylic acid benzyl ester,
2-(3,4-Dimethoxybenzyl)-9-[N-methyl-N-(3-phenylpropyl)amino]hypoxanthine,
9-[N-Methyl-N-(3-phenylpropyl)amino]-2-[4-(4-methylpiperazin-1-ylsulfonyl)benzyl]hypoxanthine,
2-(3,4-Dimethoxybenzyl)-7-[1(R)-[1(R)-hydroxyethyl]-4-phenylbutyl]-5-methylimidazo[5,1-f][1,2,4]triazin-4(3H)-one,
7-(1-Acetylpentyl)-5-methyl-2-(4-methylbenzyl)imidazo[5,1-f][1,2,4]triazin-4(3H)-one,
7-(1-Acetylhexyl)-5-methyl-2-(4-methylbenzyl)imidazo[5,1-f][1,2,4]triazin-4(3H)-one,
2-(3,4-Dimethoxybenzyl)-7-[1-(1-hydroxyethyl)-5-hexenyl]-5-methylimidazo[5,1-f][1,2,4]triazin-4(3H)-one, and
and the pharmaceutically acceptable salts of these compounds.

Salts encompassed within the term “pharmaceutically acceptable salts” refer to non-toxic salts of the compounds which are generally prepared by reacting a free base with a suitable organic or inorganic acid or by reacting the acid with a suitable organic or inorganic base. Particular mention may be made of the pharmaceutically acceptable inorganic and organic acids customarily used in pharmacy. Those suitable are in particular water-soluble and water-insoluble acid addition salts with acids such as, for example, hydrochloric acid, hydrobromic acid, phosphoric acid, nitric acid, sulfuric acid, acetic acid, citric acid, D-gluconic acid, benzoic acid, 2-(4-hydroxybenzoyl)-benzoic acid, butyric acid, sulfosalicylic acid, maleic acid, lauric acid, malic acid, fumaric acid, succinic acid, oxalic acid, tartaric acid, embonic acid, stearic acid, toluenesulfonic acid, methanesulfonic acid or 1-hydroxy-2-naphthoic acid, the acids being employed in salt preparation—depending on whether it is a mono- or polybasic acid and depending on which salt is desired—in an equimolar quantitative ratio or one differing therefrom.

As examples of salts with bases are mentioned the lithium, sodium, potassium, calcium, aluminium, magnesium, titanium, ammonium, meglumine or guanidinium salts, here, too, the bases being employed in salt preparation in an equimolar quantitative ratio or one differing therefrom.

It is understood that the active compounds and their pharmaceutically acceptable salts mentioned can also be present, for example, in the form of their pharmaceutically acceptable solvates, in particular in the form of their hydrates.

“Diseases in which pulmonary surfactant malfunction and/or phosphodiesterase 2 (PDE2) activity is detrimental” which may be mentioned are in particular disorders of varying origin. Such diseases are characterized by a pulmonary surfactant malfunction and/or an impairment of oxygenation and/or edema formation. Diseases which may be mentioned as examples are ALI (Acute Lung Injury), ARDS (Adult Respiratory Distress Syndrome), IRDS (Infant Respiratory Distress Syndrome) and Asthma bronchiale. Preferred examples are ARDS and Asthma bronchiale.

The combined use of a pulmonary surfactant and a PDE2 inhibitor or the use of a pharmaceutical composition comprising a combination an effective amount of a pulmonary surfactant and an effective amount of a PDE2 inhibitor suited for preventing or reducing the onset of symptoms of a disease, or treating or reducing the severity of a disease in a patient in need thereof, in which disease pulmonary surfactant malfunction and/or phosphodiesterase 2 (PDE2) activity is detrimental, reduces pulmonary surfactant malfunction and/or ameliorates oxygenation and/or prevents pulmonary edema formation and/or reverses pulmonary edema formation.

According to this invention, an intact and well-functioning pulmonary surfactant system is critical for normal respiration with sufficient blood oxygenation and protection from lung infection. Pulmonary surfactant is mainly comprised of phospholipids that reduce surface tension, prevent atelectasis and greatly reduce the work of breathing. The other major component consists of surfactant proteins, which optimise the biophysical function of phospholipids and/or play an important role in host defence by acting as collectins. In this context, the term “pulmonary surfactant malfunction” refers to any condition or alteration of the surfactant system that impairs the above-mentioned properties. In particular, impairment is a result of the inhibition of the surfactant activity due to infiltrated or accumulated substances in the lung, such as proteins, proteases, and debris, or aspirated or inhaled substance (e.g. acids, saltwater, meconium, or smoke), or dilution of infiltrated, accumulated, aspirated or inhaled substances. Also particular mention is made to the impairment as a result of the inhibition of surfactant activity due to impaired surfactant production, secretion, transport, assembly, and changes in its composition.

The phrase “reducing pulmonary surfactant malfunction” refers to any intervention or therapy, which partially or completely reduces the above-mentioned pulmonary surfactant malfunction and thereby partially or completely restores pulmonary surfactant function as seen in healthy humans.

According to this invention, oxygenation of blood can be determined by a method known per se and familiar to the person skilled in the art by measuring the partial oxygen pressure in the arterial blood (PaO₂) using a blood gas analyser or by the method as—for example—described by Hafner et al. [D. Häfner et al.: Effects of rSP-C surfactant on oxygenation and histology in a rat lung lavage model of acute lung injury. Am. J. Respir. Crit. Care Med. 1998, 158: 270-278]. The phrase “ameliorating oxygenation” refers to an increase in PaO₂.

According to this invention, pulmonary edema is characterized by a shift of liquid from the pulmonary vessels to the interstitial spaces and the alveolar lumen (interstitial or alveolar edema). Based on their genesis, edema may be divided in hydrostatic and permeability edema, with hydrostatic edema having cardiogenic origin (high blood pressure) and permeability edema occurring after alterations which lead to higher permeability of the endothelial and/or epithelial cell layer at the airway/vessel interface in the lung. Accordingly, the phrase “preventing pulmonary edema formation and/or reversing pulmonary edema” refers to any intervention, therapy, condition, or alteration that prevents and/or reverses partially or fully the above mentioned mechanisms of liquid-transfer from the pulmonary vessels to the interstitial spaces and the alveolar lumen and thereby prevents and/or reverses both hydrostatic and permeability edema.

The phrase “combined use” (or “combination”) embraces the administration of a pulmonary surfactant and a PDE2 inhibitor as part of a specific treatment regimen intended to provide a beneficial effect from the co-action of these therapeutic agents. Administration of these therapeutic agents in combination typically is carried out over a defined time period (usually minutes, hours, days or weeks depending upon the combination selected). “Combined use” generally is not intended to encompass the administration of two of these therapeutic agents as part of separate monotherapy regimens that incidentally and arbitrarily result in the combinations of the present invention.

“Combined use” or “combination” within the meaning of the present invention is to be understood as meaning that the individual components of the combination can be administered simultaneously (in the form of a combination medicament—“fixed combination”) or more or less simultaneously, respectively in succession (from separate pack units—“free combination”; directly in succession or else alternatively at a relatively large time interval) in a manner which is known per se and customary. As an example, one therapeutic agent could be taken in the morning and one later in the day. Or in another scenario, one therapeutic agent could be taken once daily and the other twice weekly. It is understood, that if individual components are administered directly in succession, the delay in administering the second component should not be such as to lose the beneficial therapeutic effect of the combination.

It is to be understood that present invention covers all combinations of particular and preferred aspects of the invention described herein. Thus, present invention clearly refers to all compounds or preparations mentioned herein as examples of a pulmonary surfactant and to all compounds mentioned herein as a PDE2 inhibitor and to all possible consequential combinations. In particular, combinations which may be mentioned as preferred examples of a combination of a pulmonary surfactant and a PDE2 inhibitor are
a combination of erythro-9-(2-hydroxy-3-nonyl)adenine or its pharmaceutically acceptable salts and LUSUPULTIDE,
a combination of 9-(6-Phenyl-2-oxohex-3-yl)-2-(3,4-dimethoxybenzyl)-purin-6-one or its pharmaceutically acceptable salts and LUSUPULTIDE,
a combination of 6-(3,4-Dimethoxy-benzyl)-1-[1-(1-hydroxy-ethyl)-4-phenyl-butyl]-3-methyl-1,5-dihydro-pyrazolo[3,4-d]pyrimidin-4-one or its pharmaceutically acceptable salts and LUSUPULTIDE
a combination of N-benzyl-2-(6-fluoro-2-methyl-3-pyridin-4-ylmethylene-3H-inden-1-yl)-acetamide or its pharmaceutically acceptable salts and LUSUPULTIDE
a combination of (1Z)-N-benzyl-2-(6-fluoro-2-methyl-3-(3,4,5-trimethoxybenzylidene)-3H-inden-1-yl]-acetamide or its pharmaceutically acceptable salts and LUSUPULTIDE
a combination of 4-[N-[4-[9-[N-Methyl-N-(3-phenylpropyl)amino]hypoxanthin-2-ylmethyl]phenyl]carbamoyl]piperidine-1-carboxylic acid benzyl ester or its pharmaceutically acceptable salts and LUSUPULTIDE
a combination of 4-[N-[4-[9-(N-hexyl-N-methylamino)hypoxanthin-2-ylmethyl]phenyl]carbamoyl]piperidine-1-carboxylic acid benzyl ester or its pharmaceutically acceptable salts and LUSUPULTIDE
a combination of 2-(3,4-Dimethoxybenzyl)-9-[N-methyl-N-(3-phenylpropyl)amino]hypoxanthine or its pharmaceutically acceptable salts and LUSUPULTIDE
a combination of 9-[N-Methyl-N-(3-phenylpropyl)amino]-2-[4-(4-methylpiperazin-1-ylsulfonyl)benzyl]hypoxanthine or its pharmaceutically acceptable salts and LUSUPULTIDE
a combination of 2-(3,4-Dimethoxybenzyl)-7-[1(R)-[1(R)-hydroxyethyl]-4-phenylbutyl]-5-methylimidazo[5,1-f][1,2,4]triazin-4(3H)-one or its pharmaceutically acceptable salts and LUSUPULTIDE,
a combination of 7-(1-Acetylpentyl)-5-methyl-2-(4-methylbenzyl)imidazo[5,1-f][1,2,4]triazin-4(3H)-one or its pharmaceutically acceptable salts and LUSUPULTIDE
a combination of 7-(1-Acetylhexyl)-5-methyl-2-(4-methylbenzyl)imidazo[5,1-f][1,2,4]triazin-4(3H)-one or its pharmaceutically acceptable salts and LUSUPULTIDE, and
a combination of 2-(3,4-Dimethoxybenzyl)-7-[1-(1-hydroxyethyl)-5-hexenyl]-5-methylimidazo[5,1-f][1,2,4]triazin-4(3H)-one or its pharmaceutically acceptable salts and LUSUPULTIDE.

More or less simultaneous administration of each therapeutic agent can be effected by, for example, intratracheal or intrabronchial administration to the subject in need thereof either as an instillation of the dissolved, liquid therapeutic agents, or as an aerosolised solution or as a dry powder having a fixed ratio of each therapeutic agent.

Administration of each therapeutic agent in succession, dose in time or remote in time, can be effected by any appropriate route, including, but not limited to, intratracheal or intrabronchial instillation, oral routes, intravenous routes, intramuscular routes, and direct absorption or through mucous membrane tissues. The therapeutic agents can be administered by the same route or by different routes. For example, a pulmonary surfactant may be administered by intratracheal or intrabroncheal instillation while the PDE2 inhibitor may be administered orally, intravenously, intratracheally, intrabroncheally, sublingually, intraperitoneally, or subcutaneously. The sequence in which the therapeutic agents are administered is not narrowly critical.

The most preferred route of administration of a pulmonary surfactant is the intratracheal or intrabronchial route by instillation in liquid form or as aerosolised solution or as dry powder. It is also preferred that the pulmonary surfactant is administered in form of an aerosolised solution or a dry powder by inhalation. Dry powder formulations of pulmonary surfactants are preferably prepared by the spray drying process as described in WO 9726863.

In case of intratracheal or intrabronchial administration of a pulmonary surfactant preparation, it has proven advantageous to administer suspensions or solutions of the preparations according to the invention which contain 10 to 100 mg of phospholipids per ml of suspension. Preferably, the preparations according to the invention are administered per application in such an amount that the amount of phospholipids is between 10 and 400 mg per kilogram of body weight. As a rule, administration is carried out 1 to 3 times daily over a period of 1 to 7 days. A process is preferred in which the pulmonary surfactant solution employed contains 0.5 to 2.0 mg of rSP-C (FF/I) per ml of solvent. Particular mention may be made of a process in which the pulmonary surfactant solution employed contains 0.75 to 1.5 mg of rSP-C (FF/I) per ml of solvent.

It has also proven advantageous to administer commercially available pulmonary surfactant preparations in suitable dosages in accordance with dosage regimens cited in their summaries of product characteristics. In case of intratracheal administration of BLES® the daily dose of the phospholipids will likely be in the range of 100-150 mg/kg body weight. Preferably, the daily dose will likely be 135 mg phospholipids/kg body weight. In case of intratracheal administration of CALFACTANT the daily dose will likely be 3-6 mL/kg body weight of CALFACTANT which is about 105-210 mg phospholipids and 1,95-3,90 mg Surfactant Protein-B (SP-B) per kg body weight. In case of intratracheal administration of SURFACTANT-TA the daily dose will likely be 60-120 mL SURFACTANT-TA per kg body weight. In case of intratracheal administration of PORACTANT ALFA the daily dose will likely be 100-200 mg/kg up to a daily maximum dose of 300-400 mg/kg which is about 70-280 mg phospholipids per kg body weight and 1-4 g hydrophobic proteins (Surfactant Protein-B and Surfactant Protein-C) per kg body weight. In the case of intratracheal administration of BERACTANT the daily dose will likely be 100-200 mg phospholipids per kg body weight and 4-8 mg hydrophobic proteins (Surfactant Protein-B and Surfactant Protein-C) per kg body weight. In the case of intratracheal administration of COLFOSCERIL PALMITATE the daily dose will likely be 54-162 mg phospholipids per kg body weight.

PDE2 inhibitors may be administered intraduodenally, rectally, orally, transdermally, intramuscular, subcutanously, intranasally or intravenously in doses known per se and familiar to the person skilled in the art. The most preferred route of administration of a PDE2 inhibitor is the oral route. In another preferred embodiment the PDE2 inhibitor is administered by intravenous infusion or injection. In a further embodiment the PDE2 inhibitor is administered by intramuscular or subcutaneous injection. Other routes of administration are also contemplated, including intranasal and transdermal routes, and by inhalation and by intratracheal or intrabronchial instillation.

According to U.S. Pat. No. 5,861,396, it has proved advantageous in the case of intravenous administration to administer PDE2 inhibitors in amounts of approximately 0.01 to 10 mg/kg, preferably approximately 0.1 to 10 mg/kg of body weight, to achieve effective results. In the case of oral administration of a PDE2 inhibitor, the PDE2 inhibitor may be administered in an amount known per se and familiar to the person skilled in the art depending on the type of indication and the patient in need.

In spite of this, if appropriate it may sometimes be necessary to depart from the amounts mentioned, mainly depending on the body weight or the type of administration route, on individual behavior towards the medicament, the manner of its formulation and the time or interval at which administration takes place. Thus in some cases it may be sufficient to manage with less than the abovementioned minimum amount, while in other cases the upper limit mentioned has to be exceeded. In the case of the administration of relatively large amounts, it may be advisable to divide these into several individual doses over the course of the day.

In case of pharmaceutical compositions, which are intended for oral administration, the therapeutic agent(s) are formulated to give medicaments according to processes known per se and familiar to the person skilled in the art. The therapeutic agents are employed as medicament, preferably in combination with suitable pharmaceutical carrier, in the form of tablets, coated tablets, capsules, granules, emulsions, suspensions, syrups or solutions, the therapeutic agent content advantageously being between 0.1 and 95% by weight of total mixture and, by the appropriate choice of the carrier, it is being possible to achieve a pharmaceutical administration form precisely tailored to the therapeutic agent(s) and/or to the desired onset of action (e.g. a sustained-release form or an enteric form).

The person skilled in the art is familiar on the basis of his/her expert knowledge which carriers or excipients are suitable for the desired pharmaceutical formulations. In addition to solvents, gel-forming agents, tablet excipients and other active compound carriers, it is possible to use, for example, antioxidants, dispersants, emulsifiers, antifoams, flavor corrigents, preservatives, solubilizers, colorants or permeation promoters and complexing agents (e.g. cyclodextrins).

The therapeutic agent(s) of the present invention can be administered by a variety of methods known in the art, although for many therapeutic applications, the preferred route of administration is a free combination of a pulmonary surfactant and a PDE2 inhibitor whereby the pulmonary surfactant is administered as a dry powder by inhalation or by intratracheal or intrabronchial instillation of a liquid and the PDE2 inhibitor is administered orally. For some therapeutic application it may be preferable to administer the pulmonary surfactant and the PDE2 inhibitor in a fixed combination, whereby the preferred route of administration is inhalation of a dry powder formulation or an aerosolised solution or intrabronchial instillation of a liquid formulation.

The therapeutic agent(s) are dosed in an order of magnitude customary for the individual dose. It is more likely possible that the individual actions of the therapeutic agents are mutually positively influenced and reinforced and thus the respective doses on the combined administration of the therapeutic agent(s) may be reduced compared with the norm.

Utility

Combinations of present invention may be prescribed to the patient in “patient pack” containing the whole course of treatment in a single package. Patient packs have an advantage over traditional prescriptions, where a pharmacist divides a patient's supply of a pharmaceutical from a bulk supply, in that the patient always has access to the package insert contained in the patient pack, normally missing in traditional prescriptions. The inclusion of a package insert has been shown to improve patient compliance with the physician's instructions and, therefore, lead generally to more successful treatment. It will be understood that the administration of a combination of present invention by means of a single patient pack, or patient packs of each component compound, and containing a package insert instructing the patient to the correct use of the invention is a desirable additional feature of the invention leading to an increased compliance of the patient compared to the administration of each single component.

Another beneficial effect of present invention refers to use of combinations of present invention. It has surprisingly been found that a unexpected therapeutic benefit, particularly a synergistic benefit, in the prevention or reduction of the onset of symptoms of a disease, or in the treatment or reduction of the severity of a disease in a patient in need thereof, in which disease pulmonary surfactant malfunction and/or phosphodiesterase 2 (PDE2) activity is detrimental, can be obtained by using a composition of a pulmonary surfactant and a PDE2 inhibitor.

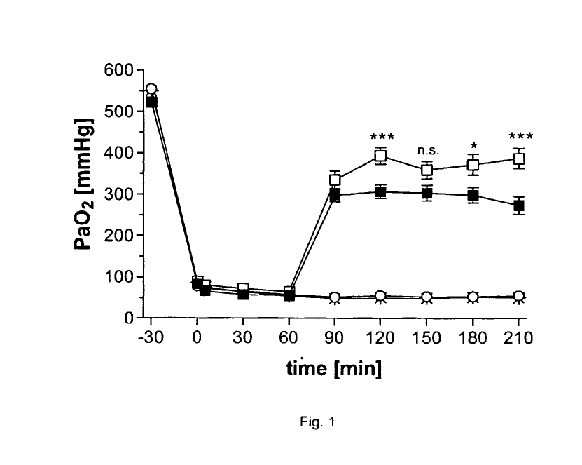

For instance, it is possible by using a combination of a pulmonary surfactant and a PDE2 inhibitor to superiorly ameliorate oxygenation in a patient in need thereof suffering from a disease in which pulmonary surfactant malfunction and/or phosphodiesterase 2 (PDE2) activity is detrimental compared to the use of a pulmonary surfactant or a PDE2 inhibitor alone. This synergistic effect of the combination of a pulmonary surfactant and a PDE2 inhibitor has been shown by in vivo studies as outlined in Example 5 and FIG. 1.

Because of this synergistic effect of a combination of present invention, the amount of the pulmonary surfactant may be significantly reduced when used in a combination with a PDE2 inhibitor, which inter alia significantly reduces costs of the therapy of a patient in need thereof, as pulmonary surfactants are comparatively costly. The frequency of ungratefulness related to the application of a pulmonary surfactant, for example, by instillation may also be reduced compared to the use of a pulmonary surfactant alone.

As another beneficial effect of a combination of present invention, there is provided as a result of the improved oxygenation a significantly improvement of patients body performance - compared to the use of a pulmonary surfactant alone or a PDE2 inhibitor alone.

Finally, it as been found that the use of a combination of a pulmonary surfactant and a PDE2 inhibitor significantly reduces the time patients with ARDS or IRDS have to be ventilated, and thus, it is possible by the administration of a combination of a pulmonary surfactant and a PDE2 inhibitor to avoid side effects of ventilation, for example the risk of a nosocomial infection or pneumonia for the patients can be lowered compared to the use of a pulmonary surfactant alone.

### DESCRIPTION OF DIAGRAMS

FIG. 1: Influence of PDE-2 inhibition and VENTICUTE administration on arterial blood oxygenation after repeated saline lung lavage in rats

Male Wistar rats were prepared according to Example 5 and lungs were ravaged 5-9 times with NaCl 0.9% (=>PaO₂˜50-100 mmHg). After 60 min NaCl 0.9% (open circles), VENTICUTE 12.5 mgPL/kg (filled squares, PL=Phospholipids), PODPO (9-(6-Phenyl-2-oxohex-3-yl)-2-(3,4-dimethoxybenzyl)-purin-6-one) 100 nM (stars), or VENTICUTE 12.5 mgPL/kg in combination with PODPO 100 nM (open squares) was administered intratracheally (administration volume 1.2 mL). Arterial blood oxygenation (PaO₂) was determined every 30 min up to 150 min after drug administration (t=210 min). Administration of NaCl and PODPO alone had no influence on oxygenation, but VENTICUTE 12.5 mgPL/kg improved oxygenation to about 300 mmHg. The combined use of both drugs, i.e. VENTICUTE 12.5 mgPL/kg and PODPO 100 nM, revealed the significant, synergistic effect of the combination in restoring oxygenation. Data are shown as mean±SEM. * p<0.05, *** p<0.001 versus VENTICUTE 12.5 mgPL/kg.

### EXAMPLES

### Example 1

### Fixed Combination LUSUPULTIDE+PODPO For Dry Powder Inhalation

9.8 g of 1,2-dipalmitoyl-3-sn-phosphatidylcholine, 4.2 g of 1-paimitoyl-2-oleoyl-3-sn-phosphatidylglyce-rolammonium, 12.3 μg of PODPO (9-(6-Phenyl-2-oxohex-3-yl)-2-(3,4-dimethoxybenzyl)-purin-6-one), 0.7 g of palmitic acid, 0.36 g of calcium chloride and 0.28 g of r-SP-C (FF/I) are dissolved in 820 ml of 2-propanol/water (90:10) and spray-dried in a Büchi B 191 laboratory spray-dryer. Spray conditions: drying gas nitrogen, inlet temperature 110° C., outlet temperature 59-61° C. A fine powder is obtained which can be micronized. About 55 mg/kg body weight can be administered intratracheally as a dry powder with an appropriate dry powder inhaler device for a single application.

### Example 2

### Fixed Combination LUSUPULTIDE+EHNA For Intrabronchial Instillation

9.8 g of 1,2-dipalmitoyl-3-sn-phosphatidylcholine, 4.2 g of 1-palmitoyl-2-oleoyl-3-sn-phosphatidylglyce-rolammonium, 0.7 g of palmitic acid, 0.36 g of calcium chloride and 0.28 g of r-SP-C (FF/I) are spray-dried as described in Example 1. 0.88 mg EHNA (erythro-9-(2-hydroxy-3-nonyl)adenine) is dissolved in 280 mL 0.9% sodium chloride. The 15.34 g of the surfactant composition are added to this solution and suspended. For a single application in humans 1 ml/kg body weight of this suspension can be instilled intrabronchially guided by a bronchoscope.

### Example 3

### Free Combination of BERACTANT For Intratracheal Instillation+PODPO For Oral Administration

For a single application in humans commercially available BERACTANT is administered intratracheally 100 mg/kg as a suspension in 0.9% sodium chloride containing 25 mg phospholipids per mL (consisting of 11.0-15.5 mg/mL disaturated phosphatidycholine, 0.5-1.75 mg/mL triglycerides, 1.4-3.5 mg/mL free fatty acids, and less than 1.0 mg/mL protein). This application is combined with one or several timed oral administrations of 1 to 20 mg PODPO [9-(6-Phenyl-2-oxohex-3-yl)-2-(3,4-dimethoxybenzyl)-purin6-one].

### Example 4

### Free Combination PORACTANT ALPHA For Intratracheal Instillation+DHPMDP For Oral Administration

For a single application in humans commercially available PORACTANT ALPHA is administered intratracheally 100-200 mg/kg. Composition per mL of suspension: phospholipid fraction from porcine lung 80 mg/mL, equivalent to about 74 mg/mL of total phospholipids and 0.9 mg/mL of low molecular weight hydrophobic proteins. This application is combined with one or several timed oral administrations of 1 to 20 mg DHPMDP (6- (3,4-Dimethoxy-benzyl)-1-[1-(1-hydroxy-ethyl)-4-phenyl- butyl]-3-methyl-1,5-dihydro-pyrazolo[3,4-d]pyrimidin-4-one).

### Example 5

### Rat Lung Lavage Experiment

Male Wistar rats (220-250 g) were anaesthetized, catheterised to withdraw arterial blood, and ventilated with pure oxygen (=>PaO₂˜500-550 mmHg). 30 min later lungs were lavaged 5-9 times with NaCl 0.9% (=>PaO₂˜50-100 mmHg). After 60 min NaCl 0.9% (open circles), VENTICUTE 12.5 mgPL/kg (filled squares, PL=Phospholipids), PODPO (9-(6-Phenyl-2-oxohex-3-yl)-2-(3,4-dimethoxybenzyl)-purin-6-one) 100 nM (stars), or VENTICUTE 12.5 mgPL/kg in combination with PODPO 100 nM (open squares) was administered intratracheally (administration volume 1.2 mL). Arterial blood oxygenation (PaO₂) was determined every 30 min up to 150 min after drug administration (t=210 min). According to FIG. 1, administration of NaCl and PODPO alone had no influence on oxygenation, but VENTICUTE 12.5 mgPL/kg improved oxygenation to about 300 mmHg. Combination of both drugs, VENTICUTE 12.5 mgPL/kg containing PODPO 100 nM, showed a significant, synergistic effect in restoring the oxygenation. Data are shown as mean±SEM. * p<0.05, *** p<0.001 versus VENTICUTE 12.5 mgPL/kg.

1. Combined use of a pulmonary surfactant and a PDE2 inhibitor for preventing or reducing the onset of symptoms of a disease, or treating or reducing the severity of a disease in a patient in need thereof, in which disease pulmonary surfactant malfunction and/or phosphodiesterase 2 (PDE2) activity is detrimental.

2. Use of a combination of a pulmonary surfactant and a PDE2 inhibitor for the preparation of a medicament for preventing or reducing the onset of symptoms of a disease, or treating or reducing the severity of a disease in a patient in need thereof, in which disease pulmonary surfactant malfunction and/or phosphodiesterase 2 (PDE2) activity is detrimental.

3. Method for preventing or reducing the onset of symptoms of a disease in which pulmonary surfactant malfunction and/or phosphodiesterase 2 (PDE2) activity is detrimental, or treating or reducing the severity of a disease in which pulmonary surfactant malfunction and/or phosphodiesterase 2 (PDE2) activity is detrimental by administering to a patient in need thereof an effective amount of (1) a pulmonary surfactant and (2) a PDE2 inhibitor.

4. The method according to claim 3, wherein an effective amount of (1) a pulmonary surfactant and (2) a PDE2 inhibitor is administered simultaneously to a patient in need thereof.

5. The method according to claim 3, wherein an effective amount of (1) a pulmonary surfactant and (2) a PDE2 inhibitor are administered in succession, close in time or remote in time, in any order whatever to a patient in need thereof.

6. Use or method according to any of claims 1 to 5, wherein the pulmonary surfactant is selected from the group consisting of PORACTANT ALFA, BERACTANT, BOVACTANT, COLFOSCERIL PALMITATE, SURFACTANT-TA, CALFACTANT, PUMACTANT, LUSUPULTIDE and SINAPULTIDE.

7. Use or method according to claim 6, wherein the pulmonary surfactant is LUSUPULTIDE.

8. Use or method according to any of claims 1 to 5, wherein the PDE2 inhibitor is selected from the group consisting of N-Benzyl-2-[5-fluoro-2-methyl-1(Z)-(3,4,5-trimethoxybenzylidene)inden-3-yl]acetamide, 2-(3′-Aminobiphenyl-4-ylmethyl)-9-(1-methyl-4-phenylbutyl)hypoxanthine, N-Benzyl-2-[5-fluoro-2-methyl-1(Z)-(3,4,5-trimethoxybenzylidene)inden-3-yl]acetamide, 2-(3′-Aminobiphenyl-4-ylmethyl)-9-(1-methyl-4-phenylbutyl)hypoxanthine, 2-Benzyl-9-(1-methyl-4-phenylbutyl)hypoxanthine, 2-(3,4-Dichlorobenzyl)-9-[1-(1-hydroxyethyl)-4-phenylbutyl]hypoxanthine, 2-(4-Fluorobenzyl)-9-(1-methyl-4-phenylbutyl)hypoxanthine, 9-(1-Methyl-4-phenylbutyl)-2-[4-(3-thienyl)benzyl]hypoxanthine, 1-[4-[9-[1-(1-Hydroxyethyl)-4-phenylbutyl]hypoxanthin-2-ylmethyl]-2-methoxyphenylsulfonyl]piperidine-4-carboxylic acid, 2-(Biphenyl-4-yl)-9-(1-methyl-4-phenylbutyl)-6,9-dihydro-1H-purin-6-one, 2-(4-Chlorophenyl)-9-[1-(1-hydroxyethyl)heptyl]-6,9-dihydro-1H-purin-6-one, 2-Cyclohexyl-9-[1-(1-hydroxyethyl)-4-phenylbutyl]-6,9-dihydro-1H-purin-6-one, 2-Cyclopropyl-9-(1-methyl-4-phenylbutyl)-6,9-dihydro-1H-purin-6-one, 2-(1,3-Benzodioxol-5-yl)-9-(1-methyl-4-phenylbutyl)-6,9-dihydro-1H-purin-6-one, erythro-9-(2-hydroxy-3-nonyl)adenine, 9-(6-Phenyl-2-oxohex-3-yl)-2-(3,4-dimethoxybenzyl)-purin-6-one, 6-(3,4-Dimethoxy-benzyl)-1-[1-(1-hydroxy-ethyl)-4-phenyl-butyl]-3-methyl-1,5-dihydro-pyrazolo[3,4-d]pyrimidin-4-one, N-benzyl-2-(6-fluoro-2-methyl-3-pyridin-4-ylmethylene-3H-inden-1-yl)-acetamide, (1Z)-N-benzyl-2-[6-fluoro-2-methyl-3-(3,4,5-trimethoxybenzylidene)-3H-inden-1-yl]-acetamide, N-Benzyl-2-[5-fluoro-2-methyl-1-[(Z)-(pyridin-4-yl)methylene]-1H-inden-3-yl]acetamide hydrochloride, 4-[N-[4-[9-[N-Methyl-N-(3-phenylpropyl)amino]hypoxanthin-2-ylmethyl]phenyl]carbamoyl]piperidine-1-carboxylic acid benzyl ester, 4-[N-[4-[9-N-hexyl-N-methylamino)hypoxanthin-2-ylmethyl]phenyl]carbamoyl]piperidine-1-carboxylic acid benzyl ester, 2-(3,4-Dimethoxybenzyl)-9-[N-methyl-N-(3-phenylpropyl)amino]hypoxanthine, 9-[N-Methyl-N-(3-phenylpropyl)amino]-2-[4-(4-methylpiperazin-1-ylsulfonyl)benzyl]hypoxanthine, 2-(3,4-Dimethoxybenzyl)-7-[1(R)-[1(R)-hydroxyethyl]4-phenylbutyl]-5-methylimidazo[5,1-f][1,2,4]triazin-4(3H)-one, 7-(1-Acetylpentyl)-5-methyl-2-(4-methylbenzyl)imidazo[5,1-f][1,2,4]triazin-4(3H)-one, 7-(1-Acetylhexyl)-5-methyl-2-(4-methylbenzyl)imidazo[5,1-f][1,2,4]triazin-4(3H)-one, 2-(3,4-Dimethoxybenzyl)-7-[1-(1-hydroxyethyl)-5-hexenyl]-5-methylimidazo[5,1-f][1,2,4]triazin-4(3H)-one, and the pharmaceutically acceptable salts of these compounds.

9. Use or method according to any of claims 1 to 8, wherein the PDE2 inhibitor is selected from the group consisting of N-Benzyl-2-[5-fluoro-2-methyl-1(Z)-(3,4,5-trimethoxybenzylidene)inden-3-yl]acetamide, 2-(3′-Aminobiphenyl-4-ylmethyl)-9-(1-methyl-4-phenylbutyl)hypoxanthine, N-Benzyl-2-[5-fluoro-2-methyl-1(Z)-(3,4,5-trimethoxybenzylidene)inden-3-yl]acetamide, 2-(3′-Aminobiphenyl-4-ylmethyl)-9-(1-methyl-4-phenylbutyl)hypoxanthine, 2-Benzyl-9-(1-methyl-4-phenylbutyl)hypoxanthine, 2-(3,4-Dichlorobenzyl)-9-[1-(1-hydroxyethyl)-4-phenylbutyl]hypoxanthine, 2-(4-Fluorobenzyl)-9-(1-methyl-4-phenylbutyl)hypoxanthine, 9-(1-Methyl-4-phenylbutyl)-2-[4-(3-thienyl)benzyl]hypoxanthine, 1-[5-[9-[1-(1-Hydroxyethyl)-4-phenylbutyl]hypoxanthin-2-ylmethyl]-2-methoxyphenylsulfonyl]piperidine-4-carboxylic acid, 2-(Biphenyl-4-yl)-9-(1-methyl-4-phenylbutyl)-6,9-dihydro-1H-purin-6-one, 2-(4-Chlorophenyl)-9-[1-(1-hydroxyethyl)heptyl]-6,9-dihydro-1H-purin-6-one, 2-Cyclohexyl-9-[1-(1-hydroxyethyl)-4-phenylbutyl]-6,9-dihydro-1H-purin-6-one, 2-Cyclopropyl-9-(1-methyl-4-phenylbutyl)-6,9-dihydro-1H-purin-6-one, 2-(1,3-Benzodioxol-5-yl)-9-(1-methyl-4-phenylbutyl)-6,9-dihydro-1H-purin-6-one, erythro-9-(2-hydroxy-3-nonyl)adenine, 9-(6-Phenyl-2-oxohex-3-yl)-2-(3,4-dimethoxybenzyl)-purin-6-one, 6-(3,4-Dimethoxy-benzyl)-1-[1-(1-hydroxy-ethyl)-4-phenyl-butyl]-3-methyl-1,5-dihydro-pyrazolo[3,4-d]pyrimidin-4-one, N-benzyl-2-(6-fluoro-2-methyl-3-pyridin-4-ylmethylene-3H-inden-1-yl)-acetamide, (1Z)-N-benzyl-2-[6-fluoro-2-methyl-3-(3,4,5-trimethoxybenzylidene)-3H-inden-1-yl]-acetamide, N-Benzyl-2-[5-fluoro-2-methyl-1-[(Z)-(pyridin-4-yl)methylene]-1H-inden-3-yl]acetamide hydrochloride, 4-[N-[4-[9-[N-Methyl-N-(3-phenylpropyl)amino]hypoxanthin-2-ylmethyl]phenyl]carbamoyl]piperidine-1-carboxylic acid benzyl ester, 4-[N-[4-[9-(N-hexyl-N-methylamino)hypoxanthin-2-ylmethyl]phenyl]carbamoyl]piperidine-1-carboxylic acid benzyl ester, 2-(3,4-Dimethoxybenzyl)-9-[N-methyl-N-(3-phenylpropyl)amino]hypoxanthine, 9-[N-Methyl-N-(3-phenylpropyl)amino]-2-[4-(4-methylpiperazin-1-ylsulfonyl)benzyl]hypoxanthine, 2-(3,4-Dimethoxybenzyl)-7-[1(R)-[1(R)-hydroxyethyl]-4-phenylbutyl]-5-methylimidazo[5,1-f][1,2,4]triazin-4(3H)-one, 7-(1-Acetylpentyl)-5-methyl-2-(4-methylbenzyl)imidazo[5,1-f][1,2,4]triazin-4(3H)-one, 7-(1- Acetylhexyl)-5-methyl-2-(4-methylbenzyl)imidazo[5,1-f][1,2,4]triazin-4(3H)-one, 2-(3,4-Dimethoxybenzyl)-7-[1-(1-hydroxyethyl)-5-hexenyl]-5-methylimidazo[5,1-f][1,2,4]triazin-4(3H)-one, and the pharmaceutically acceptable salts of these compounds.

10. Use or method according to any of claims 1 to 7, wherein the disease in which pulmonary surfactant malfunction and/or phosphodiesterase 2 (PDE2) activity is detrimental is ARDS or Asthma bronchiale.

11. Use or method according to any of claims 1 to 9, wherein the disease in which pulmonary surfactant malfunction and/or phosphodiesterase 2 (PDE2) activity is detrimental is selected from the group consisting of ALI, IRDS, ARDS and Asthma bronchiale.

12. Pharmaceutical composition suited for the use or method according to claims 1 to 8 comprising an effective amount of a pulmonary surfactant and an effective amount of a PDE2 inhibitor.

13. Pharmaceutical composition according to claim 12, comprising as a fixed combination
an effective amount of a pulmonary surfactant and
an effective amount of a PDE2 inhibitor, and optionally
a pharmaceutically acceptable carrier.

14. Pharmaceutical composition according to claim 13, which is a fixed pharmaceutical composition for intratracheally or intrabronchially instillation.

15. Pharmaceutical composition according to claim 12, comprising as a free combination
an effective amount of a pulmonary surfactant and optionally a pharmaceutically acceptable carrier and
an effective amount of a PDE2 inhibitor and optionally a pharmaceutically acceptable carrier.

16. Pharmaceutical composition according to any of claims 12 to 15, wherein the pulmonary surfactant is selected from the group consisting of PORACTANT ALFA, BERACTANT, BOVACTANT, COLFOSCERIL PALMITATE, SURFACTANT-TA, CALFACTANT, PUMACTANT, LUSUPULTIDE OR SINAPULTIDE.

17. Pharmaceutical composition according to any of claims 12 to 16, wherein the pulmonary surfactant is LUSUPULTIDE.

18. Pharmaceutical composition according to any of claims 12 to 15, wherein the PDE2 inhibitor is selected from the group consisting of N-Benzyl-2-[5-fluoro-2-methyl-1(Z)-(3,4,5-trimethoxybenzylidene)inden-3-yl]acetamide, 2-(3′-Aminobiphenyl-4-ylmethyl)-9-(1-methyl-4-phenylbutyl)hypoxanthine, N-Benzyl-2-[5-fluoro-2-methyl-1(Z)-(3,4,5-trimethoxybenzylidene)inden-3-yl]acetamide, 2-(3′-Aminobiphenyl-4-ylmethyl)-9-(1-methyl-4-phenylbutyl)hypoxanthine, 2-Benzyl-9-(1-methyl-4-phenylbutyl)hypoxanthine, 2-(3,4-Dichlorobenzyl)-9-[1-(1-hydroxyethyl)-4-phenylbutyl]hypoxanthine, 2-(4-Fluorobenzyl)-9-(1-methyl-4-phenylbutyl)hypoxanthine, 9-(1-Methyl-4-phenylbutyl)-2-[4-(3-thienyl)benzyl]hypoxanthine, 1-[5-[9-[1-(1-Hydroxyethyl)-4-phenylbutyl]hypoxanthin-2-ylmethyl]-2-methoxyphenylsulfonyl]piperidine-4-carboxylic acid, 2-(Biphenyl-4-yl)-9-(1-methyl-4-phenylbutyl)-6,9-dihydro-1H-purin-6-one, 2-(4-Chlorophenyl)-9-[1-(1-hydroxyethyl)heptyl]-6,9-dihydro-1H-purin-6-one, 2-Cyclohexyl-9-[1-(1-hydroxyethyl)4-phenylbutyl]-6,9-dihydro-1H-purin-6-one, 2-Cyclopropyl-9-(1-methyl-4-phenylbutyl)-6,9-dihydro-1H-purin-6-one, 2-(1,3-Benzodioxol-5-yl)-9-(1-methyl-4-phenylbutyl)-6,9-dihydro-1H-purin-6-one, erythro-9-(2-hydroxy-3-nonyl)adenine, 9-(6-Phenyl-2-oxohex-3-yl)-2-(3,4-dimethoxybenzyl)-purin-6-one, 6-(3,4-Dimethoxy-benzyl)-1-[1-(1-hydroxy-ethyl)-4-phenyl-butyl]-3-methyl-1,5-dihydro-pyrazolo[3,4-d]pyrimidin-4-one, N-benzyl-2-(6-fluoro-2-methyl-3-pyridin-4-ylmethylene-3H-inden-1-yl)-acetamide, (1Z)-N-benzyl-2-[6-fluoro-2-methyl-3-(3,4,5-trimethoxybenzylidene)-3H-inden-1-yl]-acetamide, N-Benzyl-2-[5-fluoro-2-methyl-1-[(Z)-(pyridin-4-yl)methylene]-1H-inden-3-yl]acetamide hydrochloride, 4-[N-[4-[9-[N-Methyl-N-(3-phenylpropyl)amino]hypoxanthin-2-ylmethyl]phenyl]carbamoyl]piperidine-1-carboxylic acid benzyl ester, 4-[N-[4-[9-(N-hexyl-N-methylamino)hypoxanthin-2-ylmethyl]phenyl]carbamoyl]piperidine-1-carboxylic acid benzyl ester, 2-(3,4-Dimethoxybenzyl)-9-[N-methyl-N-(3-phenylpropyl)amino]hypoxanthine, 9-[N-Methyl-N-(3-phenylpropyl)amino]-2-[4-(4-methylpiperazin-1-ylsulfonyl)benzyl]hypoxanthine, 2-(3,4-Dimethoxybenzyl)-7-[1(R)-[1(R)-hydroxyethyl]-4-phenylbutyl]-5-methylimidazo[5,1-f][1,2,4]triazin-4(3H)-one, 7-(1-Acetylpentyl)-5-methyl-2-(4-methylbenzyl)imidazo[5,1-f][1,2,4]triazin-4(3H)-one, 7-(1-Acetylhexyl)-5-methyl-2-(4-methylbenzyl)imidazo[5,1-f][1,2,4]triazin-4(3H)-one, 2-(3,4-Dimethoxybenzyl)-7-[1-(1-hydroxyethyl)-5-hexenyl]-5-methylimidazo[5,1-f][1,2,4]triazin-4(3H)-one, and the pharmaceutically acceptable salts of these compounds.

19. Pharmaceutical composition according to any of claims 12 to 18, wherein the PDE2 inhibitor is selected from the group consisting of N-Benzyl-2-[5-fluoro-2-methyl-1(Z)-(3,4,5-trimethoxybenzylidene)inden-3-yl]acetamide, 2-(3′-Aminobiphenyl-4-ylmethyl)-9-(1-methyl-4-phenylbutyl)hypoxanthine, N-Benzyl-2-[5-fluoro-2-methyl-1(Z)-(3,4,5-trimethoxybenzylidene)inden-3-yl]acetamide, 2-(3′-Aminobiphenyl-4-ylmethyl)-9-(1-methyl-4-phenylbutyl)hypoxanthine, 2-Benzyl-9-(1-methyl-4-phenylbutyl)hypoxanthine, 2-(3,4-Dichlorobenzyl)-9-[1-(1-hydroxyethyl)-4-phenylbutyl]hypoxanthine, 2-(4-Fluorobenzyl)-9-(1-methyl-4-phenylbutyl)hypoxanthine, 9-(1-Methyl-4-phenylbutyl)-2-[4-(3-thienyl)benzyl]hypoxanthine, 1-[5-[9-[1-(1-Hydroxyethyl)-4-phenylbutyl]hypoxanthin-2-ylmethyl]-2-methoxyphenylsulfonyl]piperidine-4-carboxylic acid, 2-(Biphenyl-4-yl)-9-(1-methyl-4-phenylbutyl)-6,9-dihydro-1H-purin-6-one, 2-(4-Chlorophenyl)-9-[1-(1-hydroxyethyl)heptyl]-6,9-dihydro-1H-purin-6-one, 2-Cyclohexyl-9-[1-(1-hydroxyethyl)-4-phenylbutyl]-6,9-dihydro-1H-purin-6-one, 2-Cyclopropyl-9-(1-methyl-4-phenylbutyl)-6,9-dihydro-1H-purin-6-one, 2-(1,3-Benzodioxol-5-yl)-9-(1-methyl-4-phenylbutyl)-6,9-dihydro-1H-purin-6-one, erythro-9-(2-hydroxy-3-nonyl)adenine, 9-(6-Phenyl-2-oxohex-3-yl)-2-(3,4-dimethoxybenzyl)-purin-6-one, 6-(3,4-Dimethoxy-benzyl)-1-[1-(1-hydroxy-ethyl)-4-phenyl-butyl]-3-methyl-1,5-dihydro-pyrazolo[3,4-d]pyrimidin-4-one, N-benzyl-2-(6-fluoro-2-methyl-3-pyridin-4-ylmethylene-3H-inden-1-yl)-acetamide, (1Z)-N-benzyl-2-[6-fluoro-2-methyl-3-(3,4,5-trimethoxybenzylidene)-3H-inden-1-yl]-acetamide, N-Benzyl-2-[5-fluoro-2-methyl-1-[(Z)-(pyridin-4-yl)methylene]-1H-inden-3-yl]acetamide hydrochloride, 4-[N-[4-[9-[N-Methyl-N-(3-phenylpropyl)amino]hypoxanthin-2-ylmethyl]phenyl]carbamoyl]piperidine-1-carboxylic acid benzyl ester, 4-[N-[4-[9-(N-hexyl-N-methylamino)hypoxanthin-2-ylmethyl]phenyl]carbamoyl]piperidine-1-carboxylic acid benzyl ester, 2-(3,4-Dimethoxybenzyl)-9-[N-methyl-N-(3-phenylpropyl)amino]hypoxanthine, 9-[N-Methyl-N-(3-phenylpropyl)amino]-2-[4-(4-methylpiperazin-1-ylsulfonyl)benzyl]hypoxanthine, 2-(3,4-Dimethoxybenzyl)-7-[1(R)-[1(R)-hydroxyethyl]-4-phenylbutyl]-5-methylimidazo[5,1-f][1,2,4]triazin-4(3H)-one, 7-(1-Acetylpentyl)-5-methyl-2-(4-methylbenzyl)imidazo[5,1-f][1,2,4]triazin-4(3H)-one, 7-(1-Acetylhexyl)-5-methyl-2-(4-methylbenzyl)imidazo[5,1-f][1,2,4]triazin-4(3H)-one, 2-(3,4-Dimethoxybenzyl)-7-[1-(1-hydroxyethyl)-5-hexenyl]-5-methylimidazo[5,1-f][1,2,4]triazin-4(3H)-one, and the pharmaceutically acceptable salts of these compounds.

20. Use of a pharmaceutical composition according to one of claims 12 to 19 for the treatment of a disease selected from the group consisting of ALI, IRDS, ARDS and Asthma bronchiale.

21. Method for preparing a pharmaceutical composition of the claims 12 to 14 comprising the step: mixing an effective amount of a pulmonary surfactant and a PDE2 inhibitor with a pharmaceutically acceptable carrier.
